(19) United States
(12) Patent Application Publication
(10) Pub. No.: US 2024/0203553 A1
(43) Pub. Date: Jun. 20, 2024

(54) OPIOID TREATMENT SYSTEM & METHOD

(71) Applicant: Andria Orlowski, Mesa, AZ (US)

(72) Inventor: Andria Orlowski, Mesa, AZ (US)

(21) Appl. No.: 18/084,318

(22) Filed: Dec. 19, 2022

(51) Int. Cl. G16H 20/10; G16H 10/20; G16H 10/60; G16H 20/60

(52) U.S. Cl. CPC G16H 20/10; G16H 10/20; G16H 10/60; G16H 20/60

(57) ABSTRACT

An improved opioid treatment system & method including a subscription-based software incorporating an array of tests to precisely and accurately determine the degree of impairment of a person's neuromodulators caused by their opioid abuse along with their overall health and condition, and then using that data to determine and provide an appropriate treatment protocol to mitigate and repair the damage caused by the abuse and restore them to their pre-drug condition.

### COPYRIGHT NOTICE

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever. 37 CFR 1.71(d).

### BACKGROUND OF THE INVENTION

### 1. Field of the Invention

This invention relates generally to the treatment of drug addiction, and more specifically to opioid treatments and methods of treating people addicted to opioids.

### 2. Description of the Related Art

Until the 1960s healthcare in this county for most Americans consisted of a general practitioner (GP) who treated diseases, set broken bones, and stitched lacerations. Very rarely was a patient referred to a specialist; that system worked well because the population was active, ate good diets, and the environment was less toxic. Nowadays the GP is contracted with various insurers or third-party payors or the practice is a large group; the days of the solo practitioner are fading. As technology improved, the wisdom of the old GP of the 1950's gave way to testing and referrals to specialists. Lately private equity has moved into healthcare in a big way buying practices. Today the average physician is answering to an accountant—type corporate superior that makes the medical decisions. The typical doctor has between 3 and 6 minutes to examine the patient, decide on a treatment plan, a memorialize the encounter on a pad or other device. For the foregoing reasons doctors simply do not have the time to allocate to tough cases. That lack of available time precludes being proficient with the various lab tests and properly analyzing the results. Often the tests ordered are inadequate or with multiple tests they may conflict.

As an actual example, a patient presented to one of the inventors. They were depressed and had been prescribed an SSRIs (Selective Serotonin Reuptake Inhibitor)—antidepressant. The inventor ordered specific lab work that established the patient's serotonin levels were already abnormally high. Thus, the antidepressant would have had a very detrimental effect. It's simply not possible for a good conscientious physician to be conversant with so many different testing protocols. Opioid abusers present an altogether different challenge that the typical physician is not equipped to deal with as in addition to the range of patient maladies they have seriously impacted their physiological systems in ways that are not readily obvious.

The embodiment is a treatment protocol built upon an AI enhanced subscription-based software that will provide to the physician a comprehensive analysis of the patient's psychological profile that considers all the relevant factors that may influence the patient's outcome. The AI function allows each patient's data and experience to add to the body of understanding thereby geometrically increasing its efficacy. Upon analysis and review of the results an individualized treatment protocol is generated and provided to the physician thereby ensuring the efficacy of the result.

America has had a love/hate relationship with opiates since the early days of the United States. President Trump declared the opioid abuse problem a crisis thereby launching the issue to the forefront of the public's consciousness. What escaped notice was this version of the opiate problem has been significant for decades. In fact, drug overdose fatalities have doubled every eight years since 1979. The Covid-19 response worsened the problem. Those in rehab programs were denied access, teenagers and young adults were precluded from attending class or working jobs. Idle time and the fear associated with the virus was the incubator to the drastic escalation of the problem. But the country has wrestled with opioid addiction problems before as set forth more fully below. The situation is complexified by the economic cauldron wherein inflation and the rampant escalation of house prices and rental costs has thrown many into the streets causing misery and financial duress, the handmaidens to drug abuse. Contemporaneously, Generation Z is the first group raised on digital devices. They are also the most anxious of any previous generation concerned about the economy, the environment, climate change, and finding meaningful purpose. This too is a breeding ground for opioid abuse which has been accelerated by the massive increase of drugs through the border. To this group a trip to the doctor usually resulted in a prescription consequently they have confidence in pills which fosters the belief if it is prescribed it must be safe therefore, I can take that pill without any adverse consequences which has driven the opioid abuse to unexpected levels among that age group. To understand the invention's genesis, it is necessary to examine the history of opioid use in the country as it one reason why the current programs have proven to be ineffectual.

An opioid is a natural, semi-synthetic, or synthetic drug that acts like opium and binds with the same cell receptors as opium. These drugs produce a narcotic effect used in anesthesia, sedation, and pain management. Drugs such as hydrocodone, Percodan, Dilaudid, fentanyl, oxycodone, and morphine are just a few of these more common drugs. Utilizing brand names and generic names create an extremely long list of possible drugs which aggravates the problem. The word Opium comes from the Greek word opus which means juice. Opium is derived from the poppy plant, Papaver somniferum. It is the source of the drugs commonly referred to as opiates or opioids.

Opiates are not new information; they have been used medicinally for 5,000 years. Organically, when the unripe seed capsule of the poppy plant is cut it produces a milky white fluid that runs out and when dried and collected is what we recognize as opium. The plant contains three significant alkaloids; Morphone, Codeine, and papaverine. The two common drugs derived from opium are Morphine and Codeine. The name Morphine comes from the Greek word Morpheus, the Greek god of dreams. This clearly establishes the drug was known and utilized by ancient people. Morphine was first separated in pure form from the plant in 1803 by a German pharmacy worker. It was hailed as a miracle cure for many ailments. In 1843 the hypodermic syringe was invented in Ireland and was subsequently introduced to this country in 1856. It expanded the drug's use geometrically. It was used to relieve pain, asthma, headaches, alcoholic's delirium tremens, gastrointestinal diseases, and menstrual cramps. It was widely utilized during the Civil War as a pain killer. It continues to be one of the most effective pain treatments. In 1830 a French physician extracted Codeine from opium. At that time the average physician did not have many prescribing choices. These drugs were viewed as being benign with nothing but good results. In fact, a Harper's Weekly cartoon during this period depicted a baby in a basinet with a bottle of opium on the table next to the child. Opium was used to calm fussy babies with many infants undoubtedly expiring from being overmedicated. Benjamin Franklin used opium to handle pain from a bladder stone. Laudanum, a mixture of opium and alcohol was prescribed generously.

Opium was also used recreationally in the late 1700's and into the 19^{th} century. Working class families in England used opium to treat all manner of illnesses. Its use was pervasive. Poppies were grown in Afghanistan and brought to China where it was traded as a commodity much like tea. In 1839 the Emperor of China became concerned by the number of opium addicts and subsequently banned the drug. The Chinese confiscated over 2 million pounds of opium owned by British East India Trading Company. That disruption led to the First Opium War with China; in other words the British went to war to maintain the flow of opium.

America's Addiction History: Both the Continental army and the British army relied upon opium to treat their wounded. Much like England opium was used recreationally and medically by all classes of people. During the Civil War the Union Army issued 10 million opium pills to its soldiers, and 2.8 million ounces of opium powders and tinctures. Many wounded soldiers became addicted. It was estimated over 400,000 soldiers were addicted by the end of the war, at the time the country's population was only 31,000,000. (By comparison the number of heroin addicts in this country in 2012 was estimated to be 181,000.) The syringe caused an explosion of Morphine addiction. Physicians over-prescribed it. By 1888, opiates comprised 15% of all prescriptions in Boston. By 1890 opiates were being sold in an unregulated market. These kinds of drugs were components in many over-the-counter preparations being sold daily. During that era women's bodies were not understood; as a consequence male doctors reached for morphine as the cure-all for female patients. Menstrual cramps, morning sickness, and “diseases of a nervous character”. By the late 1800's 60% of all opium addicts were women; that percentage was attributed to “complications of uterine and ovarian nature”. By 1896 most of the 313,000 opium addicts were women.

Throughout the 1870s and 1880s medical journals described the danger of addiction but most physicians were slow to respond to the problem and in truth their arsenal for treating diseases and complaints were limited. John Pemberton provides a striking example; he mixed cocaine and the kola nut to create coca cola as a treatment for morphine withdrawal. Heroin was first synthesized from Morphine in 1874 and was originally called diamorphine. It was given the name heroisch by Germans meaning heroic. It proved to be even more potent than Morphine but was believed to be non-addictive and therefore a good substitute for morphine. In 1898 Bayer began marketing heroin as a cough suppressant and for colds. The belief that it was an amazing substitute caused The St. James Society in 1900 to mail free heroin samples to morphine addicts to help them combat their addiction. In 1910 New York's Bellview hospital admitted its first Heroin addict; five years later the number had grown to over 400. The term “junkie” was born because the addicts scoured alleys for junk metal to sell to support their habits. By 1913 Bayer stopped manufacturing heroin. One can posit that opioids are a part of our cultural DNA passed down generation to generation. This is a contributing factor to the difficulty is dealing with this problem.

Legislation to address the escalating addiction problem began with Congress banning opium in 1905 and subsequently culminating with the Pure Food and Drug Act which was passed the next year forcing labels on medicine as many products contained these sorts of drugs. The Harrison Narcotics Tax Act of 1914 taxed opium, morphine, and cocaine. By 1924 heroin was banned domestically. This describes the transition of these drugs from legal and embraced to being outlawed.

Meanwhile on the other end of the spectrum opium smoking came to this country in earnest between 1870 and the 1910s with the influx of Chinese immigrants to build the railroads. Chinese operated ‘opium dens’ in most major cities. These were frequented by the indentured Chinese workers as well as the white American workers. This introduced the habit into the mainstream society. Between 1910 and 1920 the demographics of the drug user morphed to primarily the young street hoodlum or budding criminal. In the late 1800s the addiction problem was primarily the sick or women who had been prescribed the narcotic. Because of that demographic there was not a consensus for criminal penalties but when the typical user was a street punk, criminal, or prostitute the demand for criminal sanctions became well supported.

By 1930 the illicit heroin trade was by and large controlled by Lucky Luciano and organized crime. The favored means of taking the drug was by injection which allowed the dealer to cut the heroin with other ingredients thereby increasing profits dramatically. This made drugs a meaningful business and brought on the rampant problems associated with this sector today.

Poppies were historically grown in the “Golden Triangle” Burma (Myanmar), Thailand, and Laos. During WWII the Japanese occupied this area which interrupted the poppy supply. This prompted the United States to convince Mexico to cultivate poppies in the Sinaloa region to maintain a stable supply to treat the wounded soldiers. As an example, during the war the pilots carried first aid kits with morphine tartrate, the army issued the basic first aid kit with a gauze pad, tourniquet, and morphine. It's ironic, today Mexico is a major producer of the heroine sold illicitly domestically and yet the United States government created the Mexican supply chain.

During the 1950s the United States provided Thailand, Laos, and Burma weapons and support to fight communism thereby inadvertently arming the local warlords setting up the supply chain that has produced the majority of heroin. (Afghanistan eventually eclipsed the golden triangle as the world's major supplier.)

The epicenter of this modem crisis began with the Viet Nam War which exposed U.S. troops to massive amounts of drugs. Many soldiers returned home with a drug habit that shifted the demographics of the average user towards the white middle class population segment. It was estimated there were 750,000 addicts at the end of the Viet Nam war. This created pressure to enforce the drug laws and become more aggressive culminating with the formation of the DEA and the War on Drugs.

In 1979 China white, a new heroin which contained fentanyl, was introduced to the country. It was 50 times more powerful.

In 1992 the Columbian cartels produced a more pure version of heroin.

The second stage of the current crisis began during the 1980s when the idea of pain as a significant health issue took root and opened the door to using opiates for chronic pain management as set forth more fully below. By the 1990s opioid prescriptions had grown geometrically. This had the tendency to lessen the relevance of the illicit drugs because the prescription opioids could produce the high without the associated grief of chasing a dealer.

In 2008 Mexican poppy cultivation grew 485% and in many respects supplanted the Columbians. In 2011 Florida enforced prescription abuse laws focusing on ‘pill mills’ this drove the addicts to street drugs causing the heroin market to explode. The product was inexpensive due to the endless supply of Mexican heroin making it an easy alternative to scrambling for prescription opioids. By 2013 fentanyl appeared again. China was the source but as the market exploded Mexico began also producing it in volume. The DEA warned the crackdown on prescription drug abuse would drive abusers to heroin and fentanyl. The current belief that allowing open drug use in cities will somehow benefit the problem's solution is unsupported by historical data and has led to an explosion in crime rates and drug use.

The prescription opioid problem: The present crisis has been created by the pharmaceutical sector. For 100 years the only delivery mechanism for morphine or codeine was the syringe which limited the abuser to the IV delivery option which many would shun. In 1941 Morphine sulfate was approved by the FDA providing morphine in a pill form. In 1950 Percodan was approved by the FDA. Percodan contained oxycodone, a semi-synthetic morphine. These products were at the forefront of a wave of new opioid pain management medicine which completely transformed physicians' prescribing habits. At that time, opioids were reserved for serious health problems and cancer patients. The stigma against prescribing these medicines was pervasive with most physicians reluctant to prescribe them for their patients. That mentality changed in 1980 when a one paragraph “Letter to the Editor” published in the New England Journal of Medicine reported that of 11,882 hospital patients treated with narcotics less than 1% became addicted. That one letter was subsequently cited in 439 articles published in scientific journals, the statement was not challenged rather it had become accepted as if it were a significant study with serious critical review. Several Cornell University doctors formed the American Pain Society (“APS”) and advocated for the non-cancer pain patients to be treated with opioids, described as being safe and humane. Studies found pain was a leading health concern that was not being properly evaluated nor treated. (It was subsequently established APS and other advocacy groups received funding from Pharmaceutical companies.) Opioids presented the pharmaceutical industry with an extremely profitable niche. The focus became convincing physicians that prescribing opioids for pain was good medicine. This task was accomplished with advocacy groups and massive numbers of ‘reps’ were deployed to promote the products. A study established during 2014 and 2015 roughly 7 in 10 doctors had received a gift from a pharmaceutical company. That number was greater with primary care physicians; one in five received gifts. According to the CDC physicians wrote seventy opioid prescriptions for every one hundred patients. Studies also revealed prescribing habits were influenced by receipt of payments from pharmaceutical companies. Drug companies hired doctors to be Key Opinion Leaders and funded dinners wherein the doctor would tout the benefits of the sponsored drug to other physicians. Purdue Pharma took the sales rep concept to another level not seen before. It had a staff of 600 reps and identified doctors most likely to be receptive to their overtures.

In 1995 APS suggested that pain be evaluated on intake and categorized as ‘the 5^{th} vital sign’. APS together with the American Academy of Pain Medicine in 1997 declared there was insufficient evidence to show that addiction occurs when opioids are appropriately prescribed. The pharmaceutical companies also focused on influencing universities and academic medical centers. This was accomplished by making gifts to universities and establishing schools within a university. For example, the founding family of Perdue pharma established the Graduate School of Biomedical Sciences at Tufts University. Three years after launching OxyContin family members made a gift to establish a Masters of Science in Pain Research, Education, and Policy. Purdue also gave $3 million to Massachusetts General Hospital (‘MGH’) to create the MGH Purdue Pharma Pain Center. Purdue also made gifts to Boston University, Northeastern University, and Massachusetts College of Pharmacy among others. The Massachusetts Attorney General's complaint against Perdue included documents that revealed how Purdue's relationships with universities provided the ability to influence research, curricula, speakers, etc. The public's perception of opioids was carefully orchestrated painting the picture these medicines were safe and most probably would not be addictive.

The pharmaceutical companies influenced patient advocacy organizations, professional societies, and other such groups. Donations to these entities were instrumental in shaping public policy and legislation. A U.S. Senate report found five pharmaceutical companies donated $9 million to fourteen such groups between 2012 and 2017. Although gifts can directly create a response, often the donation or financial support generates a receptivity wherein the recipient is more favorably disposed towards the donor. A study noted corporations target their charitable donations to strategically benefit their corporate objectives. Favorable comments during a legislative initiative may result in a long term benefit. The long range goal was to completely change the perception of these drugs in both the physician and the patient.

One of the most telling examples of how far reaching these initiatives became is The National Academies of Science, Engineering, and Medicine (NASEM) which is a Non-Governmental Organization (“NGO”) created by federal statue to advise various governmental agencies on legislation. Its purpose was to ensure unbiased highly technical analysis that legislators and policy makers could rely upon. An article in BMJ (British Medical Journal) set forth concerns NASEM had received millions of dollars from pharmaceutical companies and gifts from Purdue's founding family. It also related some panel members had received research funding, consultancy fees, and advisory board retainers.

The numbers provide a compelling story. Between 2006 and 2015 pharmaceutical companies marketing opioid products spent $880 million on lobbying and campaign contributions. This concerted effort completely changed the perception of prescription narcotics ushering in the idea if the doctor prescribed it, then it must be safe and appropriate. This shift in thought is the epicenter of the opioid crisis. Hand in hand with this was the media's reaction and profiling. Studies have established the image projected by the media is drug addicts were black or of color, using IV needles, and living in the more depressed parts of a city. This image included an association with crime; the drug addicted must be engaged in criminal activity. The initial drug scourge of the late 1800's was met with very little public support for harsh laws because more often than not the addict was a middle class woman prescribed opium by her physician. When the street toughs and criminal element were using drugs the public demanded harsher laws. The prescription opioid abuser was portrayed as middle class, white, and the addiction or problem was presented as an unfortunate issue deserving sympathy. Because of that media-created image the governments were slow to crack down on the prescription opioid problem.

The merging of the prescription opioid problem and the street opioids is best exemplified by the country's most prolific bank robber, Anthony Hathaway. At 20 he began working for Boeing as a technical designer with no college education. By 30 he was an engineer making $100,000/year. He believes a game of Roller-hockey in the Boeing parking lot caused him to rupture a disc, his doctor prescribed an opioid and he became hooked.

When the prescription drugs became unavailable he turned to street drugs and began robbing banks to fund his habit. A hardworking law abiding citizen snared by the opioids was the reality of the situation which was overshadowed by his bank robbing spree. This cross-pollination of the legal prescribed drugs and the illegal opioids has fueled the current crisis. Currently counterfeit prescription pills are manufactured in Mexico and easily transported into this country. Too often the opioid appearing prescription pill is laced with fentanyl which causes severe consequences. History has established cracking down on prescription drug abuse pushes the users to street drugs. This back and forth has created the environment that fostered the current epidemic.

As more and more regular working people became addicted it generated an entirely different media perception. Statistically over ⅓ of the population has been touched by this crisis in one way or another; spouse, child, relative etc. Because of the extraordinary campaign the pharmaceutical companies implemented the thought of actual patients becoming addicted seemed farfetched in the average physician's mind. Secondarily, Purdue in particular pushed the concept that its product wasn't addictive. The narrative became the opioid that was not the problem; rather it was the patient with the problem. These dynamics overwhelmingly caught the government and NGOs flatfooted without the means to provide effective treatment protocols.

Opioid Treatment Protocols: The Cause of the opioid addiction: The standard rehabilitation protocol is built upon an abstinence premise wherein the addict is detoxified then counseled and guided for a return to life without using opioids. The prevailing treatments enjoy a 75%-90% failure rate. The existing protocols are not geared to eliminate the desire for the drug, the craving. The recidivism rate is extraordinarily high because these protocols do not touch the cause of the problem—which is also multi-faceted. The opioid crisis is caused by societal forces that have significantly changed the fabric of our lives. The direct cause of the crisis is loss of a sense of purpose, lack of a sense of community, and a widening wealth gap.

Historically drugs first came on the collective radar in the 1960s. As a point of reference many pop stars and celebrities overdosed on both illicit drugs and prescription medication, so the crisis is not a recent development. In fact, drug overdoses have doubled every nine (9) years since 1979. The only change has been the drug in favor at the time and the focus of the government's interdiction efforts.

To understand the crisis it is necessary to simply consider the dramatic changes the society has experienced since the early 1960s. As a point of reference, in the early 1960s women had limited career paths such as a wife, mother, teacher, office staff, secretary, nurse, or governmental position. There was a very rigid channeling of women's opportunities. That is of course completely different today. The massive shift in public perception over the successive decades caused turmoil and irrevocably changed the societal structure. The onset of these changes actually began with WWII. Prior to the war the country was mostly agrarian with the vast percentage of the population residing on farms, ranches, or rural settings. The war effort needed an unprecedented geometric increase in manufacturing; making war materials; guns, bombs, planes, tanks, etc. Unlike today's military, at that time when men were drafted they were gone for the duration of the war. As a consequence women were pressed into service, Rosie the Riveter is the perfect example of this shift. Women filled factory positions and management functions. this became accepted and began to pave the way for the changes experienced in the 60s.

In the 1940s the agrarian population enjoyed good diets, the food was fresh and pesticides were not that prevalent. The food did not generally contain additives or preservatives. For instance the Hostess Twinkie was manufactured in facilities around the country because they would only stay edible for a short period of time and were sold in areas close to the Hostess baking facilities. People worked hard and were generally in good physical condition. The introduction of the GI Bill put college in reach of many that never before had that opportunity. The returning veterans and their wives wanted better for their children, 30% of the baby boomers obtained a college degree, another 28% had at least some college. Higher education exposed more to a range of new ideas and experiences. With more women entering the workforce it increased the family's standard of living, suburbs were conceived, the society changed rapidly. And that dramatically affected the society. For eons children were raised by their mothers; it's how societies functioned and children learned. Basic functions like learning to read or simple societal norms were learned behavior preparing the child for school and life. With both parents working children were by necessity relegated to third party surrogates until school age and then became latchkey kids with their own key to the house. That lack of home-based learning created a disconnection that had not been present previously. In the 1940s and 50s into the early 60s children played outside and amused themselves. In the 1970s televisions became a fixture in most homes. That morphed into the videogames and ultimately the computers and pads. The society became more sedentary. With sedentary came obesity and children sitting around as opposed to running and playing outside was conducive to the drug problem. In many instances children or teenagers that have an opioid abuse problem are often enough bored.

The Viet Nam war divided the country and introduced the soldiers to drugs. The returning soldiers brought not only their drug habits but and a dramatically different perception of various drugs. The anti-war protests fueled the rebellious currents; more young people were in college with free time. Smoking pot became more accepted. The war had a sobering effect on the society. This ushered in the disco era of the 1970s which fueled the loosening of mores and drugs of all kinds entered the mainstream. As the society changed the drug of choice changed with it. The marijuana of the '60s morphed into heroine, amphetamines, Quaaludes (methaqualone), cocaine, these led to crack, meth, and oxycontin. Drug use increased, the only change was which drug was popular at each point in time. Meanwhile Wall Street discovered junk bonds and the motto was ‘greed is good’ as popularized by the movie “Wall Street”. The financiers of Wall Street held an annual dinner and targeted a venerable old company to be attacked and dismantled. This process eliminated jobs and threw people out of work. The callous attitude permeated the culture. Workers used to be proud of the job they performed. At the end of the day there was a sense of accomplishment; “Made in USA” meant something to most people and symbolized quality. Many items came with a tag that stated it had inspected for quality by a specific individual. There was a direct connection from the employee to the consumer. This has to be compared to these times with factories moving offshore eliminating workers, and the robot craze wherein as many tasks are being performed by robots and AI. The robotic intrusion into our everyday lives is pervasive. From the automated car wash to the grocery/retail stores that install self-service checkouts which eliminates employees and reduces customer interaction with the company personnel. The average American was under pressure from multiple fronts. Good jobs and careers were being eliminated, factories moved overseas, impersonal interactions in both the employment sector as well as the consumer experience, and permissiveness opened the door to drug usage. This adversely impacted the family unit. The deleterious effect on families is reflected by the statistics. A marriage in 1950 had a 20% chance of ending with a divorce; a 1970 marriage increased those odds to 50%. This resulted in a huge number of children were raised in a single-parent household. The fracturing of the family unit directly influenced these children as they grew up. Impermanence became the norm.

In 1960, 63.3% attended church; by 2004 that percentage dropped to just 17.7%. This unmoored the society there was no longer that anchored sense of community. In the past people interacted with their neighbors, the children played together. The neighbors would have BBQs or other social events. These relationships bound people with a sense of belonging. Crime rates were lower. Church attendance also created that sense of belonging, the loss of that sense of community made people less caring for one another. Homelessness grew geometrically. State operated mental health facilities became archaistic throwing many impaired individuals onto the streets and increased drug problems.

In the 1965 the average company CEO earned 20 times what the company's typical worker earned; by 1989 that ration became 58 to 1, and presently it is 278 to 1. From 1978 CEO to 2018 compensation rose 1,005%. The typical worker's salary went up just 12% since 1978. John D. Rockefeller was the country's first billionaire in the early 1900s. In 1982 Forbes magazine published its first list of the 400 richest Americans. The net worth necessary to make that list was $91 million ($225 million in today's dollars). The 2018 list required $2.1 billion to make the list. In 1982 there were just 13 billionaires; today there are 560. The richest 1% in the country own more than the bottom 90% of the population. This disparity has created the dynamic for the raging opioid crisis. The current attitude towards a job is called “quiet quitting”. Previously people worked hard and strived for career advancement. Now, the trend is to do as little as possible to retain the employment but view it as a paycheck and nothing more.

The 2008 financial crisis completely changed homeownership in this country. Foreclosed homes were bought up by hedge funds and people who had owned their homes transitioned into tenants. Rents escalated forcing people to double up or children to move in with their parents and in the worst cases pushed people onto the streets. The push for higher education began in the 1960s and accelerated over the successive decades, which has resulted in far more colleges and universities—with an explosion of tuition costs. Would-be and attending students were encouraged to take out loans, saddling many graduating seniors with massive debt and few job opportunities that will produce income sufficient enough to repay it. The prevailing metric today has been for Private Equity firms to buy up housing thereby artificially raising prices with the idea to transition the country to renters. This breaks up the American Dream of homeownership and belonging in a neighborhood.

These societal forces were the cause of the opioid crisis. The foreclosures subsequent to 2008 spawned a host of problems. Crushing student debt without the hope of ever finding employment sufficient to service the debt was depressing at best and led to a myriad of consequences. These issues manifested in the opioid crisis itself. In 2011 oxycodone was the drug most commonly found in overdose deaths. Stricter enforcement of prescription drug abuse drove users into the illicit market. That result is very clear; from 2012-2015 heroin surpassed prescription drugs as the major cause of overdose death. Recently, it has been shifting towards fentanyl due to its cost and availability. In just a few years the problem morphed again.

The cause of the typical programs lackluster results. The prevailing treatment system generally claims success rates of up to 30%—a figure many experts view as dubious. But if we accept that at face value, by the industry's own admission 70% of people who enter rehab programs come out no better off for the experience. From the foregoing it is evident the traditional treatment protocols do not address the underlying causes of the addiction which are societal. This succinctly explains the failure rate. The core of the problem is the addiction creates an insatiable desire to use the substance which, unchecked, leads to a craving. Without removing the cause of that desire, one is forever chained to that potential craving. The present systems teach the consequences of a relapse and all the bad things that a drug addiction entails—that fear drives the individual to abstain. Unexpected stresses can and do cause relapses which explains the recidivism rate.

Accordingly, the present invention and protocol considered the causes, the inherent inadequacies of the existing treatments, and focused on two areas; eliminating the desire and restoring the abuser's body and mind to a pre-addiction condition.

Accordingly, the present invention overcomes the disadvantages associated with prior methods of treating opioid addiction, by providing an improved opioid treatment system & method.

### BRIEF SUMMARY OF THE INVENTION

In view of the foregoing disadvantages inherent in the known types of opioid treatments and methods or the like, the present invention provides an improved opioid treatment system & method. As such, the general purpose of the present invention, which will be described subsequently in greater detail, is to provide an improved opioid treatment system & method that is constructed upon an AI enhanced subscription-based software incorporating an array of tests to precisely and accurately determine the degree of impairment of a person's neuromodulators caused by their opioid abuse along with their overall health and condition, and then using that data to determine and provide an appropriate treatment to mitigate and repair the damage caused by the abuse and restore them to an optimized pre-drug condition, with all the advantages of the prior art treatments and methods and none of the disadvantages, attaining a state of health which allows the patient to maintain a more balanced neurotransmitter profile to minimize tendencies of addictive behavior.

There has thus been outlined, rather broadly, the more important features of the invention in order that the detailed description thereof that follows may be better understood and in order that the present contribution to the art may be better appreciated.

Numerous objects, features and advantages of the present invention will be readily apparent to those of ordinary skill in the art upon a reading of the following detailed description of presently preferred, but nonetheless illustrative, embodiments of the present invention when taken in conjunction with the accompanying drawings. The invention is capable of other embodiments and of being practiced and carried out in various ways. Also, it is to be understood that the phraseology and terminology employed herein are for the purpose of descriptions and should not be regarded as limiting.

### BRIEF DESCRIPTION OF THE DRAWINGS

The figures which accompany the written portion of this specification illustrate embodiments according to the teachings of the present invention.

FIG. 1 shows a simplified flow chart of the method of using the opioid treatment system within the preferred embodiment of the present invention. The Provider is able to follow the simple process of administering the provided questions and tests, emailing the scanned results to the program which are processed by our software, sorted and scanned for abnormalities, comparing the evidence of obvious cofactor need with Genetic factors, and analyzing for potential contra indicators. This information goes though AI analysis producing recommendations which are then reviewed for efficacy by our physician, the finalized protocol is transmitted back to the provider to implement with the patient.

FIG. 2 shows a flowhart illustrating a simplistic representation of the process the client goes through the software of the instant invention performs according to the preferred embodiment of the present invention.

FIG. 3 illustrates neurotransmitters being directly affected by diet, genetic dispositions, and environmental factors.

FIG. 4 illustrates the usual addiction cycle of a person.

FIG. 5 is the original algorithm the inventors utilized to begin building the treatment protocol that highlights the interconnectedness of the various elements, testing, and external factors that come into play in the addiction cycle.

FIG. 6 is a more detailed block diagram of the range of testing aspects of the protocol and their interrelatedness which demonstrates the complexified nature of the testing and in particular how the protocol can be precisely individualized.

FIG. 7 and FIG. 8 are block diagrams of a hypothetical patient with the connected lines indicating the testing results obtained and the individualized treatment protocol.

The various embodiments of the present invention will hereinafter be described in conjunction with the appended drawings.

### DETAILED DESCRIPTION

The embodiments of the present disclosure described below are not intended to be exhaustive or to limit the disclosure to the precise forms disclosed in the following detailed description. Rather, the embodiments are chosen and described so that others skilled in the art may appreciate and understand the principles and practices of the present disclosure.

The following embodiments and the accompanying drawings, which are incorporated into and form part of this disclosure, illustrate embodiments of the invention and together with the description, serve to explain the principles of the invention. To the accomplishment of the foregoing and related ends, certain illustrative aspects of the invention are described herein in connection with the following description and the annexed drawings. These aspects are indicative, however, of but a few of the various ways in which the principles of the invention can be employed and the subject invention is intended to include all such aspects and their equivalents. Other advantages and novel features of the invention will become apparent from the following detailed description of the invention when considered in conjunction with the drawings.

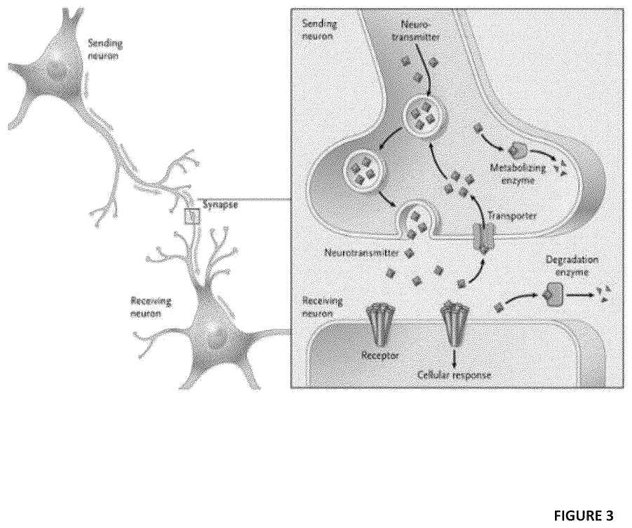

Too often in medicine the manifestation of the disease becomes the focal point in lieu of discerning the actual cause of the disease. This is the root cause of the poor results in treating opioid disorders. Not enough has been known about the actual cause of addictions. This gap in knowledge became the epicenter of the inventors' inquiry. There are multitudes of studies proving that poor nutrition and imbalances in the endocrine and metabolic processes are markers of opioid abusers. The endocrine imbalances and damage to the HPA access caused by opioid abuse results in adrenal insufficiency, blood sugar dysregulations, and hypogonadism. These imbalances together with genetic dispositions and irregularities in the neurotransmitters are a contributing cause of the poor treatment outcomes. As illustrated in FIG. 3, the cofactors of neurotransmitters are directly affected by diet, genetic dispositions, and environmental factors.

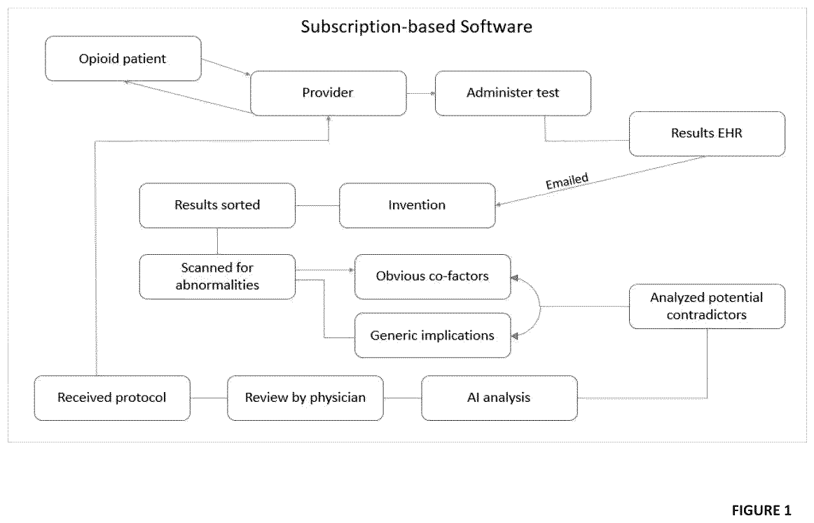

The invention includes of two parts. The first is an AI enhanced subscription-based software FIG. 1 constructed upon an innovative medical protocol that relies upon an array of sophisticated questionnaires, tests and examination to precisely and accurately determine the impairment of the neuromodulators and the overall health and condition of the abuser which is the second component. From that data it determines and provides the appropriate cofactor to mitigate the damage caused by the abuse as well as preexisting factors, genetic and lifestyle based, that may have prompted the abuser initially to seek drugs and develop an addiction. In order to restore the addict to a balanced pre-drug condition. The inventors opted for a subscription-based software to ensure the continuity of care and to make the protocol available to a wider group of providers. The uniqueness of this software is indicated by the fact there is nothing like it presently in the market.

There is wealth of research across the spectrum of human-centric related matters; the missing link is often that research grants are micro-specific and, as a consequence, the findings have not been condensed and more importantly applied to a greater benefit. The invention is focused upon findings of functional medicine that noted striking attributes of the abuser's genetic and metabolomic profile that when adequately measured with sophisticated testing was revelatory. Restoring the natural balance of these systems with the appropriate cofactors mitigates or eliminates the desire or craving for the opioid. Without the craving and desire the addiction is contained and treatment outcomes are successful. These testing processes and resultant individualized cofactors have been utilized to successfully treat an array of other diseases and disorders and so the opioid inquiry became a logical extension of observed previous patient experiences.

Genetic predispositions, gut microbiome, and neural circuits effect on the opioid abuser is little understood and current therapies are targeted towards treating the manifestation (the addiction) and not succinctly tracing the cause which has been the contributing agent in the dismal results at effectively treating this epidemic. The interaction of these systems is the basis for many of the body and mind characteristics. On account of such a broad range of impact these systems have research into these areas is extensive yet not necessarily directed at discerning the cause of the opioid addiction; hence a gap in the knowledge. The inventors' continuing investigation into these systems as related to many disorders including opioid misuse gave rise to the quantification of the same into an algorithm to both standardize the treatment protocol and become the basis for software which takes these numerous factors into account by using AI to run cross analysis on the results and format the results to user friendly recommendations that can be scaled to provide a much greater impact on the crisis.

In treating the opioid abuser certain traits and patterns emerged. Creating a specific testing format to illuminate the condition of the neural circuits, identify genetic tendencies, and measure the individual's nutritional abnormalities is crucial to treating the opioid abuser. The epicenter of the abuser's dilemma is the desire or craving to ingest the substance. The body experiences withdrawal causing the mind to become restless and driven to obtain the drug. The inventors focused on the cause of that desire which is the root of the epidemic; solve that riddle and the problem can be resolved. Measuring the nutritional health and imbalances in the endocrine and metabolic processes illuminates the health of the subject. The imbalances and/or damage to the HPA access, implicates adrenal insufficiency and other inadequacies. These imbalances need to be resolved or it will adversely impact the success of an outcome. Genetic dispositions and irregularities in the neurotransmitters are also data points in the algorithm. The purpose is to make it simple for any provider to administer the battery of tests the inventors found to be effective to arrive at a good treatment protocol. Opioid abusers focus on obtaining the drug, obtaining the funds to pay for it, and associate with other users discarding their friends. In that process good nutrition and any reasonably healthy lifestyle more often than not recedes in the rear view mirror. The abuser's body must be reasonably healthy and balanced or it implicates any effective protocol.

Previous experience has demonstrated the root cause of the addiction is the desire or craving which can be mitigated or entirely eliminated with the appropriate testing protocol and addition of the indicated cofactor(s). This is an innovative approach that the investigators have developed over time and are unaware of any other such active research in this sector. A survey of patent filings confirmed the inventors' protocol is unique.

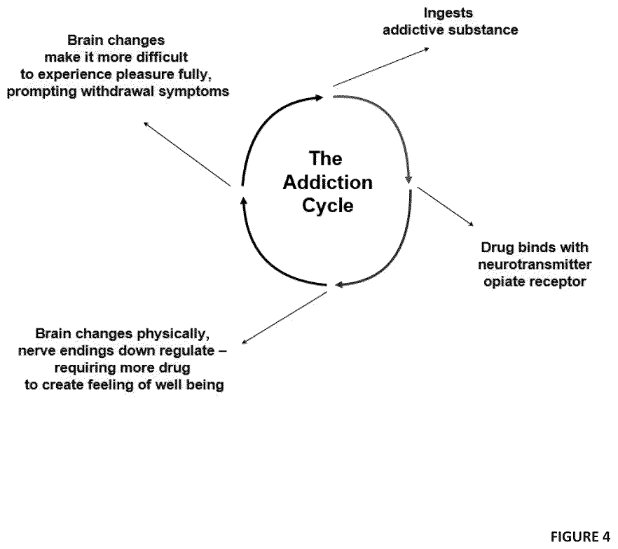

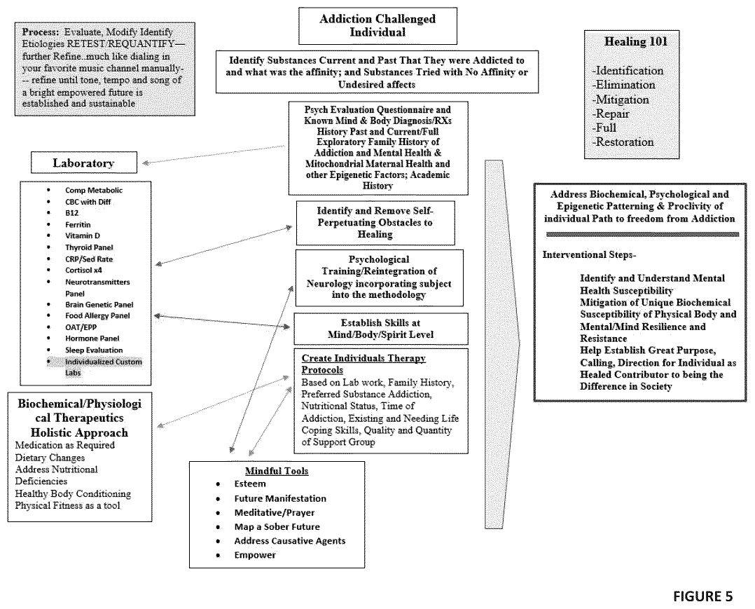

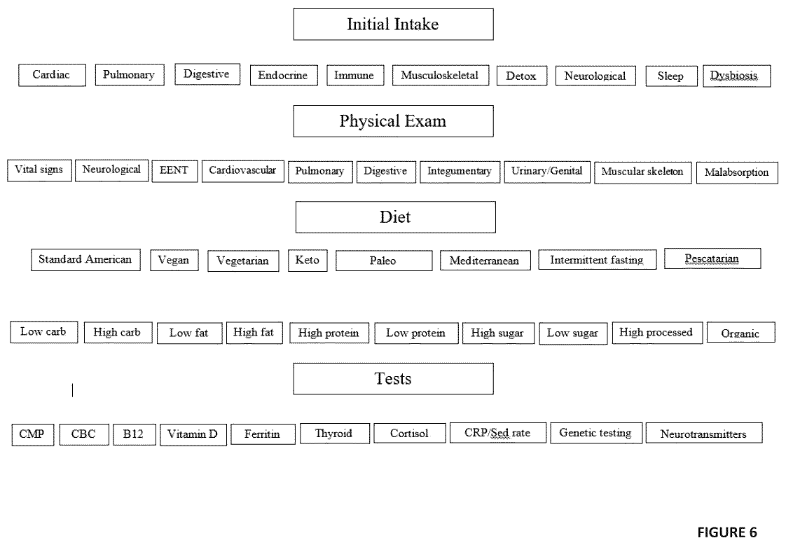

The basic chart, shown in FIG. 4, depicts the addiction cycle. The opioid binds with the neurotransmitter opiate receptor. This stimulates physical changes within the brain. Over time the nerve endings down regulate which causes the abuser to perceive a need to increase the dosage. This process impacts the reward center which precludes the ability to enjoy normal pleasures and thus the cycle increases eventually leading to the overdose. The inventors have found testing the condition of these pathways always reveals an imbalance and when corrected restores the neural transmitters to pre-abuse metrics. This is fundamental to the invention as the first step in the typical treatment protocol is to induce the abuser to cease using opioids. This can come about in a variety of ways but results with the abuser clean and sober. The typical second step is to initiate a counseling protocol. Most programs are in-residence wherein the abuser resides at the facility to facilitate medical monitoring, observation, and proper support. In adjudicated cases the abuser is remanded to a halfway house wherein they reside with other abusers, must get a job, and at some point released and expected to maintain their sobriety. The cornerstone of these treatment protocols is abstinence; mind over matter with the knowledge and perspective of the life's damage deemed to be the deterrent. Other protocols utilize Suboxone or Methadone and similar drugs to interdict the physical need for the Opioid. The foregoing has proven to be ineffective. The lynchpin is the inability to control the desire or craving which subsequently overpowers reason. The addiction cycle may appear to be dormant until stress, a calamity, or the craving becomes intolerable pushing the struggling abuser into the cycle again. None of the prevalent programs measure, quantify, or examine the subject's overall health, their deficiencies, or the condition of the neurotransmitters. Compounding the treatment problems is the fact that 29% of those with mental health problems abuse drugs or alcohol. Often the abuse is an attempt to self-medicate the effects of depression or anxiety. The invention's software anticipates this abuser subset and will accommodate the appropriate steps to ascertain the existence of this factor and the most viable response to mitigate it. (This scenario illustrates why the recidivism rates are so high with traditional programs, an individual suffering from anxiety would have an impossible task to stay sober without mitigating the anxiety which often has an organic dysfunction within the brain.)

Images of brain scans have demonstrated the substantial differences between a normal brain and an abuser's brain. These are precisely what the inventors discovered which led to the design of this invention and its software application. Without providing the correct cofactors to allow the brain to restore its normal configuration a treatment protocol is doomed to failure. An analogy would be requiring an individual with a broken leg to run the 50 yard dash and then being disappointed when they don't finish.

There are various factors that characterize the addiction challenged individual and the various steps necessary to bring about healing. These factors and steps are essential within the inventors software program and are as follows:

a. Process: Evaluation, Modify, Identify Etiologies—retest and quantify—further refine as warranted. This process accurately expresses the overarching purpose of the protocol and its software and reflects the protocol the investigators have developed and utilized. The software by design will allow the provider to individualize the treatment protocol without having to go to extraordinary measures. Basically, the design is like fine tuning a radio with the traditional dial that one can rotate for the best reception. By minutely testing the subject's various bodily systems the practitioner can discern the system's performance and identify the condition of the neural pathways.

b. Addiction Challenged Individual intake process. In the traditional treatment protocol or the typical medical practice driven by the reimbursement scheme the provider is under tremendous pressure to see as many patients as possible in an hour and peck away on a pad to fill in the Electronic Health Records which precludes listening to the patient. Ironically the inventors noted the patient is a font of information that points to their inherent physical condition and the stated affinity for a particular drug illuminates the condition of their neurotransmitters and neural functions. For instance the average methamphetamine user has an iron deficiency which in part drives the craving for the drug. For these reasons the intake is the starting point as it directs the provider to the specific testing that may be necessary to diagnosis and treat the subject. During this process the subject's mental health and known abnormalities are chronicled which further directs the inquiry and testing protocol.

c. Identify and Remove self-perpetuating obstacles to healing. The abuser has suffered a serious blow to their esteem and self-perception particularly if they had a good productive life before the addiction and allowed it to consume their life. Reversing that process while resetting the neural transmitters is crucial to prevent unnecessary diversions.

d. Addiction Challenged Individual enhanced intake process. As previously mentioned in the traditional treatment protocol or the typical medical practice funded by third party payers, the provider is under tremendous pressure to see as many patients as possible in an hour and peck away on a pad to fill in the Electronic Health Records which crowds out listening to the patient. A JAMA study found 60% to 80% of patients are not forthcoming or candid with their physician; 33% feared being judged, a larger subset found the provider is not listening as they tap their pad taking notes. The inventors transcended that barrier by prescreening and scheduling lengthy initial visits and use comprehensive questionnaires which are reviewed with the patient. When the patient feels listened to they provide the sort of information that points to their inherent physical condition. The software “Artificial Intelligence” will flag answers that warrant further investigation. Discerning their affinity for a particular drug illuminates the condition of their neurotransmitters and neural functions we will subsequently find after testing. The inventors are thorough and nonjudgmental.

e. Laboratory testing. The intake process illuminates the testing the inventors utilize with the subject; (this is proprietary work product) the focus on individualizing the testing to capture the impediments to balancing the neurotransmitters. As expressed in more detail in the software section the tests and results have many subsequent alternative choices which the software will identify and suggest. For example, often one test result will indicate further testing to establish the true health of the subject. The connection between results and additional testing has been developed over years of work. Current EMR programs advise a doctor while examining a patient for a stubbed toe he could also bill for examining the ankle and so forth with the idea to make that simple office visit more lucrative. In this case the software will advise the doctor certain results will indicate they should conduct further testing or prescribe a cofactor. By design the software is consistent with what the physician is already familiar with.

f. Biochemical/Physiological Therapeutics Holistic Approach. This is delineated into four (4) categories; a) medication as required, b) dietary changes, c) Address Nutritional Deficiencies, and 4) Healthy Body Conditioning (teaching the subject to properly care for themselves.)

g. Healing 101. The healing phase is built upon five (5) key points: a) Identification-properly and accurately understanding the mental health susceptibility, b) Elimination—shedding unproductive traits and habits, c) Mitigation—map the formulae for the subject to counter personal impediments to healthy mental wellbeing, d) Repair—adding the cofactors to address the subject's organic imbalances, e) Full Restoration which spans mind-body, and spirit for a well-rounded resilient individual. Unequivocally the abuser has lost touch with any semblance of a purpose in their life; the Rolling Stones song Ruby Tuesday properly expresses that predicate; “Catch your dreams before they slip away, dying all the time, lose dreams and you will lose your mind”. The human being is a creature of hope when hope which drives dreams are crushed the individual becomes unhinged which spawns addictions. Very much restoring the subject's natural predilections towards productive life is a cornerstone to a drug-free future. Obviously the inventors have further developed these ingredients together with the biological aspects of our protocol.

The Subscription-Based Software: The Problem the software solves: Before the advent of the onboard computer systems when a vehicle developed a problem a mechanic had to diagnose the problem. That was unpredictable at best with many a misdiagnosis. Once computers and chips played a bigger role diagnosing became simple and precise. With today's vehicles the condition that causes a check engine light to appear on the dashboard can be readily identified with a stop at an auto parts store. Connecting a small handheld computer to the vehicle quickly and precisely identifies the part that needs to be replaced. Diagnosing ailments in a human being is too often inaccurate. Analyzing the opioid abuser is far more complicated because of the changes the drug has imposed on the body. This is precisely why addiction treatment protocols are so ineffectual. Without concisely determining the condition of the abuser's various physical and mental health the sobriety protocol breeds failure. In many instances the abuser suffers from a mental health issue or a vitamin or amino acid deficiency that drives them to self-medicate. As an example, a mother brought her 14-year-old daughter to see one of the inventors with the complaint of Premenstrual dysphoric disorder (PMDD) [often incorrectly referred to as “PMS” ]. Her condition was so severe both her mother and the patient were concerned about the girl's well-being. Upon analysis the doctor recognized she had an amino acid deficiency and prescribed the appropriate one which cleared up the problem immediately. That succinctly illustrates a physical ailment that could easily drive the individual to self-medicate and ultimately addiction and demonstrates precisely why this invention is so timely and unique.

The average doctor allocates 6 minutes per patient. It is impossible in that timespan to understand the range of health issues a patient may have and most certainly is inadequate to accurately assess the various indicators this invention measures. The AI by design is constantly updating its database which will provide a software that is not available in the market. The patients' data is inputted which is correlated and ranked inclusive of the ultimate treatment protocol. The typical Electronic Health Records programs physicians utilize includes pop up suggestions for items the doctor can bill for during the visit. For example, if the patient has stubbed their toe the pop up will advise the doctor, they can also bill for examining the ankle or toes and so forth during the visit. The invention will present the provider not only the suggested protocol but will also present the efficacy percentage of the protocol as well as insight into how other patients fared when exhibiting similar attributes. For difficult cases this provides the physician with the tools that have already been proven to have benefitted a similar patient. This greatly increases the likelihood of a good outcome and limits wasted effort. In other words, every patient result enriches the database and is readily available to the next provider. This is not available in the market today. The inventors anticipate this software format will also lend itself to other maladies.

The invention's software will not only accurately diagnosis the patient's condition but also direct the provider to the appropriate remedy. In the old Star Trek television show Spock was seen playing three-dimensional chess. In a similar manner these charts are represented in two dimensional when in the real world this process is three dimensional. The human being is three-dimensional, and any effective protocol must be holistic to encompass the entire being.

The initial interview by design determines whether the individual is motivated to overcome their addiction and if they have the support system necessary to achieve a good result. Hippocrates specified the three elements to a healing: 1) the physician, 2) the art (medicine), and 3) the patient. Without the patient participating there cannot be a healing. Many programs include those that have been adjudicated or pushed to attend by family but lack self-imposed motivation. The protocol requires a motivated genuine patient which the intake interview will distinguish.

The initial intake includes the patient completing an extensive specific questionnaire focusing on their health and physical and mental wellbeing. The doctor then reviews that with the patient and expands on the results which are then digitized. The topics cover the listed bodily systems such as cardiac, pulmonary, digestive, and so forth. This process identifies glaring obvious issues that would need to be examined to verify the candidate is physically and mentally able to participate in the protocol.

The physical exam examines the listed nine (9) categories. The objective is to assess the patient's overall health and isolate areas that may need further investigation.

Diet “Der Mensch ist, was er iβt′” (you are what you eat) is a famous quotation from the German philosopher Feuerbach. Presently the cutting edge of medicine is the gut biome as it has been found to directly influence the brain activity. Drug abusers have extremely poor diets that adds to the patient's difficulties.

Each aspect of the foregoing is inputted into the software for the program to evaluate and manipulate.

The testing. This is again an oversimplification of the range of tests utilized in this protocol. It must be borne in mind the tests are in a three-dimensional array wherein one links with another and so on. The displayed tests are a fraction of the testing that might be utilized on a patient depending upon their needs and what the process reveals. The software automatically correlates each. Each category has multiple subcategories and if one indicates an abnormally high or low measurement it triggers other testing or analysis.

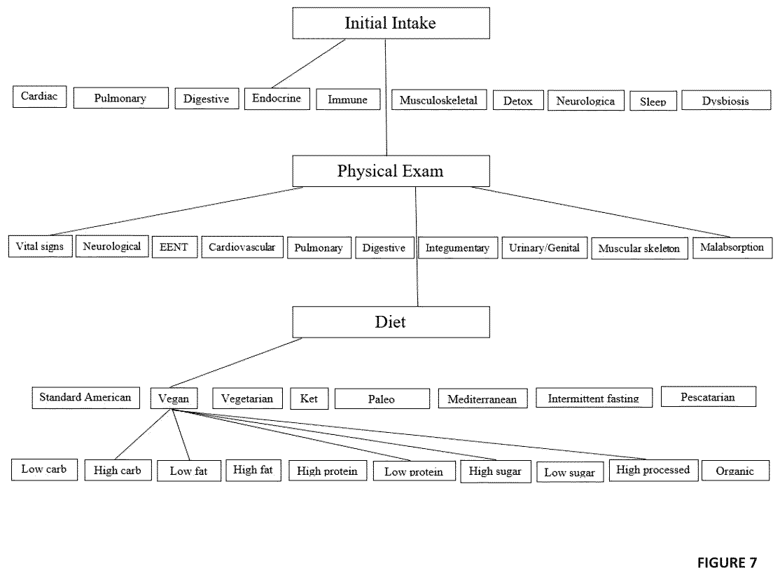

The Hypothetical Patient: FIG. 7 presents a hypothetical patient and the resulting test scores. Initial Intake: A 19-year-old female, lives with parents, and using opiates for one and a half years. Started after being given hydrocodone subsequent to wisdom teeth extraction. She found it helped her anxiety and continued to seek drugs. Then, she started college but dropped out as she was overwhelmed, anxious and couldn't focus. She went to a rehab program but was back on drugs in three weeks as she couldn't cope without them. She now works part time and feels drugs have ruined her life. She has a supportive family.

Office visit and Physical Exam: Patient suffers from insomnia and anxiety. Family history of anxiety on mother's side. Physical exam: Blood pressure low with mild orthostatic hypotension, nails ridged and spooning, pale complexion, but otherwise normal.

Dietary Evaluation: Vegan for two years. Diet high in simple carbohydrates, processed foods and high fructose corn syrup. Low protein intake. No nutritional supplementation. No signs of food allergies.

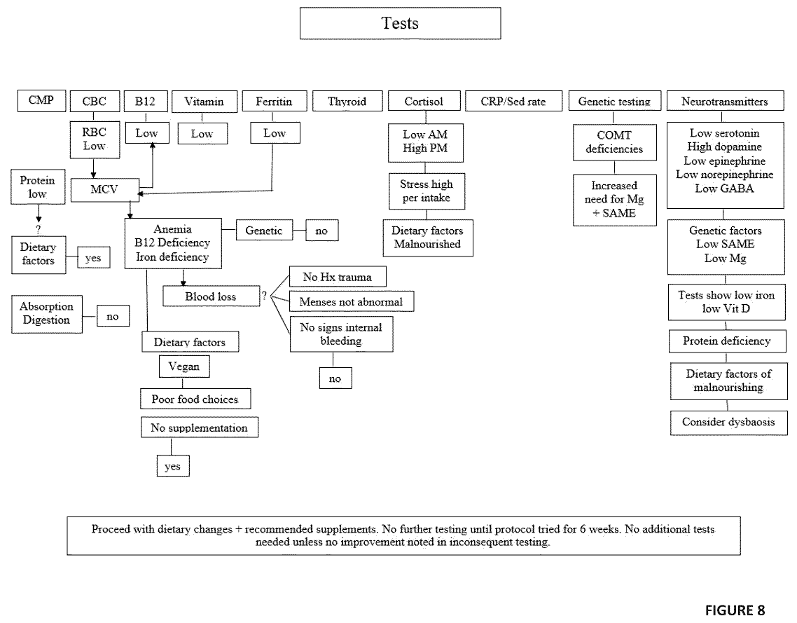

Tests: CBC-low hematocrit, low RBC, elevated MCV CMP-low protein/albumin. B12-low, vitamin D-low, Ferritin-low, thyroid-WNL, Cortisol x4-Cortisol low in AM, elevated at night. CRP/SED Rate-Average inflammation. Genetic Screen—COMT deficiencies, MTHFR one gene allele, Neurotransmitter Screen—Low Serotonin, Dopamine, PEA, Norepinephrine and GABA Elevated Glutamate. FIG. 8 shows a flow chart of testing.

Protocol: Dietary changes: increase protein, decrease simple carbohydrates, eliminate processed foods, add fresh vegetables. Supplementation: Iron with Vitamin C, B complex which includes Methylcobalamine, and MTHF, 5-HTP Magnesium, and Ashwaganda to balance Cortisol and reduce anxiety.

The objective of the invention is to widely spread this protocol and thereby have the maximum effect in treating those so afflicted. The invention is a subscription-based software format that the subscriber utilizes in treating the opioid abuser. Together with the software is a series of test kits that as set forth in the flow chart each patient would be administered. Because as previously covered each patient is unique therefore the testing becomes individualized. For this reason the invention is subscription based wherein different providers administer the tests provided to them. The tests are analyzed by the assigned labs with the individual results forwarded to the inventors for analysis to better assist the provider in individualizing the protocol. This generates uniformity while allowing for the uniqueness of each patient's complexity. Testing kits and training are supplied to the providers. This allows the healing protocol to be decimated on a much broader scale. The Inventors' AI (Artificial Intelligence) software analyzes the results and in the process learns to identify as-yet-known traits and predilections which then becomes incorporated in the software. This process constantly improves the protocol which increases the efficacy while creating the maximum benefit for the patient.

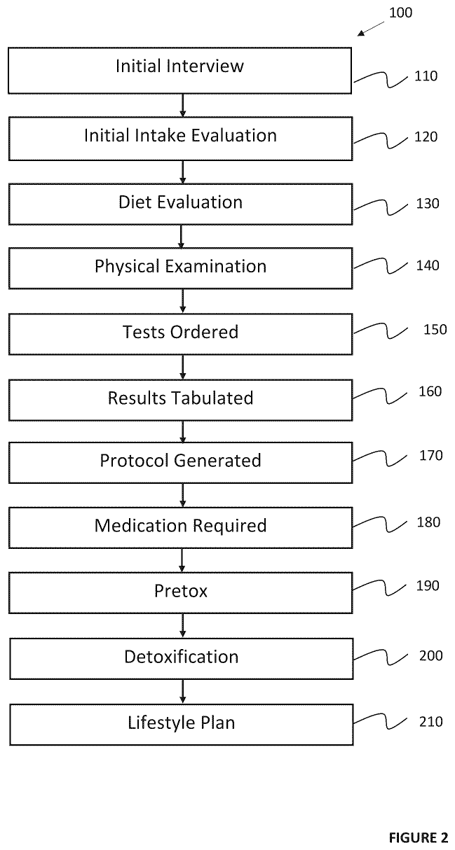

FIG. 2 is a flowhart which is a simplistic representation of the process Note 100 the software performs within the improved opioid treatment system & method. The invention's medical protocol working with the patient and their health issues that very directly affects the success rate of any abuse interdiction program include the following steps:
1. An Initial Interview Note 110: In order to qualify questions are designed to determine that the client has the necessary desire and motivation to change and that an adequate social support system is in place to be able to benefit from the protocol. Desire and motivation will be quantified and qualitatively evaluated to capture willingness and ability to put in the time, effort and compliance to glean the optimal impact of the program at the time of the questionnaire; understanding life's complexity when accounted for allows for maximum outcomes relative to patient slots available.
2. Initial Intake Evaluation Note 120: A comprehensive questionnaire that includes Psychological Evaluation, known mind and body diagnosis. Prescription history past and current, full exploratory family history of addiction, mental health, mitochondrial maternal health and other epigenetic factors as well as academic history. Answers provide information that is divided into categories that illuminate potential genetic predilections, neurological susceptibilities and nutritional imbalances that can be further evaluated and considered. Individualized approach for addressing genetic and biochemical variation is literally the key to a customized approach for each participant. Results are statically evaluated by the software based upon our criteria to determine whether or not the candidate can psychologically and physically benefit from the protocol. For those that qualify the provider is presented with a customized evaluation tool.
3. Targeted Diet Evaluation Note 130: Drug utilization can contribute and aggravate pre-existing nutritional deficits altering ability to address physical and neurological (mental) “catch-22” predisposition to self-medication. The gut microbiome directly affects the neurotransmitters and modulators. Absorption issues must be factored in and addressed to truly have the desired impact.
4. Physical Examination Note 140: Comprehensive physical exam targeting areas flagged from Initial Intake. The purpose is to determine any physiological hurdles to successfully benefiting from our program. At this time concerns that are flagged from our intake form can be discussed and further explored. A traditional physical exam of the skin, oral cavity, cardio-pulmonary system, GI, and the liver help provide nutritional status. Additional a through nutritional physical exam will be conducted as well.
5. Tests Ordered Note 150: The following tests are done on all patients:
   a. Comprehensive Metabolic Panel. Includes markers that if outside of range point to physical imbalances that contribute to fatigue, pain, and altered nutritional cellular status:
      i. Glucose
      ii. Sodium/Potassium
      iii. Kidney Liver Function
      iv. Calcium and CO2
      v. Protein and Albumin
   b. Complete Blood Count with Differential: CBC will point to iron, B12, and folate status, also ability to oxygenate brain and body. Also, offer insights into the immune status.
   c. B12: Low B12 increases risk of psycho-neurological and hematological symptoms and destabilizing DNA.
   d. Vitamin D: Low levels have been linked to opioid dependency.
   e. Ferritin: Low ferritin increases energy deficits that often is self-medicated and critical for Red blood cell and thus oxygen status.
   f. Thyroid Panel: Thyroid status directly impacts the risk for depression or anxiety, as well as the ability to think clearly and detoxify. As well as metabolism properly prescribed medications.
   g. Cortisol x4: Adrenal production of cortisol helps maintain blood sugars (prevent hypoglycemia a risk factor for addiction), and also controls pain and mood.
   h. CRP/Sed Rate: Measures of inflammation provides essential insights as to the inflammatory source of pain, also neuro-inflammatory dysregulation that contributes to altered psychological status, depression, anxiety, and decreased ability to think clearly.
   i. Genetic Screen: Numerous genetic SNPs/Polymorphisms increase susceptibility to addiction and when treated can minimize the genetic fueling and lessen susceptibility to addiction.
   j. Neurotransmitters: Measures metabolites of neurotransmitters. This demonstrates patters of imbalance that often drives patients to self-medicate with drugs. Fifty-two % of addicts have a preexisting untreated psychological condition that causes to self-medicate seeking relief. Paired with nutritional and genetic variations specific nutritional deficiencies are determined.
6. Results Tabulated Note 160: Software generated report will suggest follow-up tests that need to be run to clarify more specific protocol variations. The software has been custom created to identify and weight risks contributing to biochemical, psychosocial, and genetic susceptibility. Combined with laboratory test results, physical exams, and psychological status examination, an individualized protocol is generated to meet the participant where they are at the beginning of the program; and is used to reassess throughout successful completion.
7. Protocol Generated Note 170. Specific tailored supplementation created to address specific deficiencies and metabolic needs due to genetic variation and current health status. Self-medication(s) that have been used, duration, nutritional status, and laboratory tests all quantify dosing, route of administration, and duration of nutritional interventions.
8. Medication Required Note 180: There is a time and place for properly prescribed medications, notably to lessen withdrawals, stabilize mood, and address physical conditions that medically/ethically need to be addressed regardless of addiction history. As participants progress, medications, and nutritional interventions will be continuously monitored and adapted to the progress or newly identified needs.
9. Pretox Note 190: Supplementation and dietary changes are implemented to prepare the client for successful detoxification. By ensuring that the balanced nutritional cofactors are on board a more successful detoxification can be ensured. Proper nutrition can stabilize the individual as drug users neglect their dietary needs in favor of feeding their addiction. This precursor to detoxification eliminates the potential pitfalls and failings of the drug detoxification process.
10. Individualized Detoxification Note 200. (The precise process implemented will be the subject of a separate patent application.) The drugs are eliminated in accordance with our protocol through a controlled safe environment concurrent with administration of specific nutrients and/or cofactors that are individualized to the client's genetic and nutritional needs and variations.
11. Lifestyle Plan Note 210: Having successfully detoxified from the drug, the patient is integrated into our rehabilitation program, and life style changes and the implementation of proactive routines are established that are healthy and life sustaining including life coping skills, healthy eating, body conditioning, meditation and rewarding work habits.

As shown in the flowchart of FIG. 1, the instant software has been custom created to identify and weigh risks contributing to biochemical, psychosocial, and genetic susceptibility. Combined with laboratory test results, physical exams, and psychological status examination, an individualized protocol is generated to meet the participant where they are at the beginning of the program; and is used to reassess throughout successful completion.

Although specific embodiments have been illustrated and described herein, it will be appreciated by those of ordinary skill in the art that any arrangement, which is calculated to achieve the same purpose, may be substituted for the specific embodiment shown. This application is intended to cover any adaptations or variations of the present invention.

Although the invention has been explained in relation to its preferred embodiment, it is to be understood that many other possible modifications and variations can be made without departing from the spirit and scope of the invention.

What is claimed is:

1. An opioid treatment method for an individual comprising the steps of:
providing an initial interview with said individual;
wherein said initial interview is adapted to determine whether said individual is motivated to overcome their addiction and if they have the support system necessary to achieve desired results;
providing an initial intake;
wherein said initial intake is adapted to obtain information from said individual, including cardiac, pulmonary, and digestive system information, in order to identify possible medical issues that would need to be examined to determine if said individual is able to be positively affected by said treatment;
providing a dietary evaluation;
wherein said dietary evaluation is adapted to accumulate information pertaining to said individual's dietary habits;
providing a physical exam;
wherein said physical exam is adapted to assess said individual's overall health and isolate areas that may need further investigation;
providing testing;
wherein said testing is adapted to accumulate medical information of chosen physical and mental areas of said individual;
tabulating results;
wherein said tabulating results is adapted to use said information obtained from said testing and dietary evaluation to create a protocol of medicinal, physical, and dietary instructions for said individual to follow to achieve said desired results;
creating said protocol; and
providing said protocol;
wherein said protocol is provided to said individual to follow to achieve said desired results.

2. The opioid treatment method of claim 1, wherein said initial interview includes the following categories:
determining the willingness of said individual to allocate the necessary time, effort, and compliance to achieve optimal impact of a determined protocol; and
determining whether the individual understands the complexities of following a determined protocol.

3. The opioid treatment method of claim 1, wherein said initial intake includes the following categories:
a psychological evaluation;
a mind and body diagnosis;
a prescription drug history;
a family history of addiction;
a family history of mental health;
a family history of mitochondrial factors;
a family history of maternal health factors;
a family history of epigenetic factors; and
said individual's academic history.

4. The opioid treatment method of claim 1, wherein said dietary evaluation includes the following categories:
nutritional deficits;
gut microbiome; and
gut absorption.

5. The opioid treatment method of claim 1, wherein said physical exam includes the following categories:
skin;
oral cavity;
cardio-pulmonary system;
gastro-intestinal; and
liver

6. The opioid treatment method of claim 1, wherein said testing includes the following categories:
a. a comprehensive metabolic panel, including markers that point to physical imbalances that contribute to fatigue, pain, and altered nutritional cellular status, including:
i. glucose
ii. sodium/potassium
iii. kidney liver function
iv. calcium and CO2
v. protein and albumin;
b. complete blood count with differential: CBC will point to iron, B12, and folate status, also ability to oxygenate brain and body, also offer insights into the immune status;
c. B12: wherein low B12 increases the risk of psycho-neurological and hematological symptoms and destabilizing DNA;
d. vitamin D: wherein low levels have been linked to opioid dependency;
e. ferritin: wherein low ferritin levels increases energy deficits that often is self-medicated and critical for red blood cell and thus oxygen status;
f. thyroid panel: wherein thyroid status directly impacts the risk for depression or anxiety, as well as the ability to think clearly and detoxify, as well as metabolizing prescribed medications;
g. cortisol x4: wherein adrenal production of cortisol helps maintain blood sugars (prevent hypoglycemia a risk factor for addiction), and also controls pain and mood;
h. CRP/Sed Rate: wherein measures of inflammation provides essential insights as to the inflammatory source of pain, also neuro-inflammatory dysregulation that contributes to altered psychological status, depression, anxiety, and decreased ability to think clearly;
i. genetic screen: wherein numerous genetic SNPs/Polymorphisms increase susceptibility to addiction and when treated can minimize the genetic fueling and lessen susceptibility to addiction; and
j. neurotransmitters: which measures metabolites of neurotransmitters, and demonstrates patters of imbalance that often drives patients to self-medicate with drugs, wherein nutritional and genetic variations specific nutritional deficiencies are determined.

7. The opioid treatment method of claim 1, wherein said protocol includes the following categories:
medications;
wherein said medications are adapted to lessen withdrawal symptoms, stabilize mood, and address physical conditions.

8. The opioid treatment method of claim 1, further comprising the step of:
providing pretox;
wherein pretox is adapted to prepare said individual for detoxification;
wherein pretox includes supplementation and dietary changes to ensure proper nutrition, which can stabilize said individual as drug users usually neglect their dietary needs in favor of feeding their addiction.

9. The opioid treatment method of claim 8, further comprising the step of detoxification.

10. The opioid treatment method of claim 9, further comprising the step of:
providing a lifestyle plan;
wherein said lifestyle plan includes life coping skills, healthy eating, body conditioning, meditation, and rewarding work habits.

11. The opioid treatment method of claim 10, further comprising the step of:
providing follow-up examinations;
wherein said follow-up examinations are adapted to monitor said individual and determine if said protocol is effective, and if changes to said protocol are necessary; and
when changes are necessary to said protocol, determining which changes are necessary.

12. The opioid treatment method of claim 1, wherein said step of tabulating results is aided by the use of computer software adapted to identify and weigh risks contributing to biochemical, psychosocial, and genetic susceptibility of said individual; wherein said software is adapted to determine the degree of impairment of a person's neuromodulators caused by their opioid abuse along with their overall health and condition; and wherein when combined with said tabulating results said protocol is generated.
